(19) United States
(12) Patent Application Publication
(10) Pub. No.: US 2010/0304409 A1
(43) Pub. Date: Dec. 2, 2010

(54) METHOD FOR DETECTING PREMATURE SENESCENCE IN TUMOR CELLS AND A KIT FOR DETECTING PREMATURE SENESCENCE IN TUMOR CELLS

(75) Inventors: Jae-Seon LEE, Seoul (KR); Kee-Ho LEE, Seoul (KR); Hae-Ok BYUN, Yeongdong-gun (KR); Na-Kyung HAN, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: KOREA INSTITUTE OF RADIOLOGICAL & MEDICAL SCIENCES, Seoul (KR)

(21) Appl. No.: 12/472,135

(22) Filed: May 26, 2009

(51) Int. Cl. G01N 33/53

(52) U.S. Cl. 435/7.92; 435/7.1

(57) ABSTRACT

The present invention provides a method for detecting senescence in tumor cells and a kit for detecting senescence in tumor cells. eEF1A1 and CD markers are provided, wherein changes in the levels of the markers are correlated with premature senescence of tumor cells.

### BACKGROUND OF THE INVENTION

1. Field of the Invention

The present invention relates to premature senescence in tumor cells, in particular, methods and kits to detect premature senescence in tumor cells.

2. Description of the Related Art

Cellular senescence was originally described in normal human cells undergoing a finite number of divisions before permanent growth arrest. Cells undergoing such replicative senescence are metabolically active and exhibit distinct morphologic changes and phenotypic markers. Cellular senescence has recently become regarded as a general biological program of terminal growth arrest because a variety of treatments have been shown to trigger cellular senescence. The activated ras or raf oncogenes trigger senescence in normal cells, and low doses of DNA-damaging agents, including ionizing radiation (IR) and chemotherapeutic drugs, induce senescent phenotypes in cancer cells. Accumulating evidences suggest that apoptosis may not be the exclusive or even the primary mechanism underlying loss of self-renewal capacity in IR or drug-treated cancer cells. Recent studies suggest that induction of premature senescence is a promising treatment for solid tumors (Serrano M. Cancer regression by senescence. N Engl J Med 2007;356: 1996-7; Campisi J. Suppressing cancer: the important of being senescent. Science 2005;309:886-7).

The characteristic phenotypes of premature senescence are abundant in premalignant neoplastic lesions. Premature senescence is not only a barrier to tumorigenesis but also a hallmark of premalignant tumors. Therefore, senescence markers could be useful diagnostic and prognostic tools (Collado M, Serrano M. The power and the promise of oncogene-induced senescence markers. Nat Rev Cancer 2006;6:472-6).

The most commonly used senescence biomarker is senescence associated β-galactosidase (SA-h-Gal; Dimri G P, Lee X, Basile G, et al. A biomarker that identifies senescent human cells in culture and in aging skin in vivo. Proc Natl Acad Sci USA 1995;92:9363-7). Senescence-associated heterochromatic foci are also considered senescence markers (Collado M, Serrano M. The power and the promise of oncogene-induced senescence markers. Nat Rev Cancer 2006;6:472-6).

However, these markers have the disadvantage of being unable to be detected using standard immunohistochemical methods. Recently, DNA microarray analysis identified the oncogene-induced premature senescence markers p15INK4b, DCR2, and DEC1 (Collado M, Gil J, Efeyan A, et al. Tumor biology: senescence in premalignant tumors. Nature 2005;436:642). However, senescence markers in action are still a few.

Although premature senescence may also occur in normal cells, it is more important in that premature senescence can be used for anticancer therapy particularly in tumor cells.

Because, radiotherapy and chemotherapy, major regimens of cancer treatment, recently have been proved to induce premature senescence (or accelerated senescence) in tumor cells (Gewirtz D A, Holt S E, Elmore L W. Accelerated senescence: an emerging role in tumor cell response to chemotherapy and radiation. Biochem Pharmacol 2008; 76:947-57). Therefore, specific markers for the detection of premature senescence in tumor cells that have diagnostic and prognostic value await identification.

Moreover, tumor cells will undergo cellular senescence as well as other final cell fates (i.e., apoptosis, autophagy, etc.) following a variety of treatments such as radiation and chemotherapeutic drugs. For more accurate studies and therapeutic applicability of premature senescence, there is a need for development of biomarkers and detection methods capable of accurately distinguishing premature senescence from other final cell fates, which will provide a beneficial tool for study and cultivation of tumor cells.

### SUMMARY OF THE INVENTION

The present invention is directed to a biomarker or biomarkers for detecting premature senescence in tumor cells, wherein the biomarker is selected from eukaryotic elongation factor 1 alpha 1 (eEF1A1) and cathepsin D (CD).

In one aspect, the present invention provides a method for detecting premature senescence in tumor cells, including (a) detecting a level of a target protein in a tumor sample, wherein the protein is one or more selected from the group consisting of eEF1A1 (eukaryotic elongation factor 1 alpha 1) and CD (cathepsin D); and (b) comparing a level of the target protein of Step (a) and a level of the same protein in a control tumor cell sample to determine one or more of decrease of eEF1A1 level and increase of CD level in the tumor sample.

In another aspect, the present invention provides a method for detecting a premature senescence state of tumor cells in a human subject, comprising: (a) detecting a level of a target protein in a tumor sample obtained from the human subject, wherein the protein is one or more selected from the group consisting of eukaryotic elongation factor 1 alpha 1 (eEF1A1) and cathepsin D (CD); and (b) comparing a level of the target protein of Step (a) and a level of the same protein in a control tumor cell sample to determine one or more of decrease of eEF1A1 level and increase of CD level in the tumor sample.

In yet another aspect, the present invention provides a method for the diagnosis or prognosis of a premalignant or malignant state of tumor cells in a human subject, comprising: (a) detecting a level of a target protein in a tumor sample obtained from the human subject, wherein the protein is one or more selected from the group consisting of eukaryotic elongation factor 1 alpha 1 (eEF1A1) and cathepsin D (CD); and (b) comparing a level of the target protein of Step (a) and a level of the same protein in a control tumor cell sample to determine one or more of decrease of eEF1A1 level and increase of CD level in the tumor sample.

In yet another aspect, the present invention provides a method of screening for a premature senescence-inducing agent in a tumor cell, comprising: (a) collecting a first tumor cell sample before exposing to a candidate premature senescence-inducing agent; (b) collecting a second tumor cell sample after exposing to a candidate premature senescence-inducing agent; (c) detecting levels of one or more of CD and eEF1A1 in the first and second samples; (d) determining whether a level of CD increases and/or whether a level of eEF1A1 decreases; and (e) identifying a candidate agent exhibiting one or more of an increased CD level and a decreased eEF1A1 level, as a premature senescence-inducing agent.

In yet another aspect, the present invention provides a method of screening a potential candidate of cancer therapy in a human subject, the method comprising: (a) collecting a first tumor cell sample before exposing to a candidate premature senescence-inducing agent; (b) collecting a second tumor cell sample after exposing to a candidate premature senescence-inducing agent; (c) detecting levels of one or more of CD and eEF1A1 in the first and second samples; (d) determining whether a level of CD increases and/or whether a level of eEF1A1 decreases; and (e) identifying a potential cancer therapy agent exhibiting one or more of an increased CD level and a decreased eEF1A1 level, as a premature senescence-inducing agent.

In one embodiment of the present invention, the tumor sample is tumor cells or tumor tissues. The tumor cells or tumor tissues are derived from a mammal, preferably a human.

In another embodiment of the present invention, the control tumor cell sample is non-senescent tumor cells, and the non-senescent tumor cells are, for example, tumor cells without treatment of irradiation or drugs, that is, untreated tumor cells and are derived from a mammal, preferably a human.

In another embodiment of the present invention, levels of CD and eEF1A1 are detected using antibodies that specifically bind to these proteins. A preferred example of the antibody-based level determination is Western blot analysis. Specifically, the comparison of the protein levels is performed by measuring and comparing the intensity of bands according to Western blot analysis with naked eyes or image scanning analysis.

In another embodiment of the present invention, the premature senescence is one or more selected from irradiation (or IR)-induced senescence and anticancer drug-induced senescence, and the anticancer drug is preferably camptothecin, etoposide, and/or doxorubicin.

In another embodiment of the present invention, the control tumor cell sample is tumor cells prior to application of irradiation or an anticancer drug, and the tumor sample is tumor cells after application of irradiation or an anticancer drug.

In another embodiment of the present invention, the control tumor cell sample is tumor cells prior to application of irradiation, and the tumor sample is tumor cells 3 to 5 days after application of irradiation.

In another embodiment of the present invention, the control tumor cell sample is tumor cells prior to application of an anticancer drug, and the tumor sample is tumor cells 3 to 5 days after application of an anticancer drug.

In the context of the present invention, the tumor cells are specifically selected from breast cancer cells, lung cancer cells, colon cancer cells, and any combination thereof Further, the present invention provides a kit for detecting premature senescence, including antibodies directed against eEF1A1 and antibodies directed against CD.

### BRIEF DESCRIPTION OF THE DRAWINGS

These and other features, aspects, and advantages of the present invention will be apparent from the following detailed description, appended claims, and accompanying figures where;

FIG. 1 shows a cell picture taken on Day 4 after irradiation exposure of the MCF7 cell line (A), and the results of colony formation assay (B), respectively.

FIG. 2 shows a staining picture of senescence-associated β-galactosidase activity of the MCF7 cell line according to days after irradiation exposure thereof FIG. 3 shows a graph illustrating quantitative analysis results for the senescence-associated β-galactosidase activity of the MCF7cell line on the basis of days after irradiation exposure thereof.

FIG. 4 shows two-dimensional protein electrophoretic patterns for the comparison of protein bodies (A) and protein spots with alterations of levels in irradiation-induced senescent cells (B, C).

FIG. 5 shows Western blotting results to confirm changes in levels of CD and eEF1A1 in the irradiation-induced senescent cell line (MCF7).

FIG. 6 shows a staining picture of the senescence-associated β-galactosidase activity of senescent cells in response to irradiation exposure of the irradiation-induced senescent cell line (H460) (A); Western blotting results confirming changes in levels of eEF1A1 and CD in response to the progress of senescence (B); a staining picture of the senescence-associated β-galactosidase activity of senescent cells in response to irradiation exposure of the irradiation-induced senescent cell line (HCT116) (C); and Western blotting results confirming changes in levels of eEF1A1 and CD in response to the progress of senescence (D).

FIG. 7 shows micrographs of cells confirming senescence or apoptosis of a tumor cell line (MCF7) in response to treatments of doxorubicin at a high dose (10 μg/mL) and a low dose (50 ng/mL), respectively.

FIG. 8 shows a staining picture of the senescence-associated β-galactosidase activity for confirming senescence of a tumor cell line (MCF7) in response to treatment of low-dose doxorubicin (50 ng/mL) (A); and Western blotting results confirming changes in levels of eEF1A1 and CD of a tumor cell line (MCF7) in response to treatments of doxorubicin at a high dose (10 μg/mL) and a low dose (50 ng/mL), respectively (B).

FIG. 9 shows micrographs of cells confirming senescence or apoptosis of a tumor cell line (H460) in response to treatments of doxorubicin at a high dose (10 μg/mL) and a low dose (50 ng/mL), respectively (A); and Western blotting results confirming changes in levels of eEF1A1 and CD of a tumor cell line (H460) in response to treatments of doxorubicin at a high dose (10 μg/mL) and a low dose (50 ng/mL), respectively (B).

FIG. 10 shows Western blotting results confirming changes in levels of eEF1A1 and CD (A); and a staining picture of the senescence-associated β-galactosidase activity (B), upon treatments of a breast cancer cell line (MCF7) with camptothecin and etoposide.

FIG. 11 shows Western blotting results for understanding the relationship between transient cell cycle arrest and level changes of eEF1A1 and CD in a breast cancer cell line (MCF7).

FIG. 12 shows Western blotting results for understanding the relationship between autophagy and level changes of eEF1A1 and CD in a breast cancer cell line (MCF7) (A); and microscopic observations of cell morphology with tamoxifen-induced autophagy (B).

### DETAILED DESCRIPTION OF THE INVENTION

The present invention relates, in part, to a newly discovered biomarker for the detection of premature senescence in tumor cells. The biomarker is a eukaryotic translation elongation factor 1 alpha 1 (NCBI Access No. BC071619) protein(eEF1A1) and/or cathepsin D (NCBI Access No. BC016320) protein (CD).

Upon occurrence of premature senescence in tumor cells, a level of eEF1A 1 decreases and a level of CD increases, as compared to non-senescent cells (for example, a tumor cell sample without treatment of irradiation or drugs, that is, control or untreated or non-senescent tumor cells). Therefore, whether tumor cells undergo senescence can be determined by detecting changes in levels of eEF1A1 and/or CD.

Therefore, the present invention provides a biomarker for detecting premature senescence in tumor cells.

The premature senescence-detecting biomarker may be used for a variety of applications, such as selection of senescent cells upon culture of tumor cells, determination of therapeutic effects of radiotherapy or chemotherapy on malignant tumor cells, and screening of chemotherapeutic drugs having anticancer activity due to senescence induction.

In one aspect, the present invention provides a method for detecting premature senescence in tumor cells, including (a) detecting a level of a target protein in a tumor sample, the protein being one or more selected from the group consisting of eEF1A1 (eukaryotic elongation factor 1 alpha 1) and CD (cathepsin D); and (b) comparing a level of the target protein of Step (a) and a level of the same protein in a control tumor cell sample to determine one or more of decrease of eEF1A1 level and increase of CD level in the tumor sample. As used herein, the term “control tumor cell sample” refers to a treatment-naive tumor cell sample which was not subjected to treatments of irradiation or chemotherapeutic drugs.

The tumor sample is, for example, tumor cells or tumor tissues. The tumor cells or tumor tissues are preferably derived from a mammal, and more preferably from a human. In one preferred embodiment, the cells and the tissues are isolated from a sample obtained from a patient. In one embodiment, the sample is selected from the group consisting of biopsy and surgical samples.

As used herein, the term “cell” is intended to encompass a cell obtained from mammal and a cell line etc. The cell line refers to commercially available cells used for purposes of studies in laboratories.

The term “increase” means that a level of CD is higher in a tumor sample(for example, an irradiation or drug-treated senescent tumor cell) than in a control tumor cell sample (for example, a tumor cell sample without treatment of irradiation or drugs, that is, control or untreated or non-senescent tumor cells). For example, a level of CD is 1, 2, 3, 4, 5, 6, 7, 8, 9 or 10-fold, preferably 2.24-fold higher in the tumor sample, as compared to the control tumor cell sample.

The term “decrease” means that a level of eEF1A1 is lower in a tumor sample(for example, an irradiation or drug-treated senescent tumor cell) than in a control tumor cell sample.

For example, a level of eEF1A1 is 0.9, 0.8, 0.7, 0.6, 0.5, 0.4, 0.3, 0.2, 0.1 or 0.01-fold, preferably 0.73-fold lower in the tumor sample, as compared to the control tumor cell sample.

In the method of the present invention, the control tumor cell sample is non-senescent cells, and a preferred example of the non-senescent cells is a tumor cell without treatment of irradiation or drugs, that is, control or untreated tumor cells. The non-senescent cells are derived preferably from a mammal, and more preferably a human. In one preferred embodiment, the cells are isolated from a sample obtained from a patient. In one embodiment, the sample is selected from the group consisting of biopsy and surgical samples.

The term “non-senescent cells” refers to cells capable of undergoing cell division (actively dividing cells). The non-senescent cells do not exhibit characteristics of senescent cells, for example morphological changes of cells into flat cell shapes, senescence-associated β-galactosidase activity at a certain pH (pH 6.0), accumulation of lipofuscin (autofluorescent lysosomal pigment), formation of senescence-associated heterochromatic foci, non-responsiveness of immediate early genes to growth factors, changes of gene expression (with no occurrence of transcription), and/or increased expression levels of p53, p21, p16 and other cyclin-dependent kinase inhibitors (e.g., p27 and p 15).

The term “senescent cells” refers to non-dividing cells, which have above-mentioned characteristics intrinsic to the senescent cells.

In the method of the present invention, levels of eEF1A1 and/or CD may be detected by any conventional method known in the art (see, e.g., Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992).

Preferably, levels of eEF1A1 and CD are detected using antibodies that specifically bind to these proteins. Preferred is Western blot analysis.

In the method of the present invention, specifically the comparison of protein levels between the protein of Step (a) and the same protein in a control tumor cell sample may be performed by measuring and comparing the intensity of bands according to Western blot analysis with naked eyes or image scanning analysis. With this method, qualitative and quantitative detection of the protein can be made.

In the method of the present invention, the premature senescence is preferably one or more selected from irradiation-induced senescence and anticancer drug-induced senescence.

In the method of the present invention, the control tumor cell sample is preferably tumor cells prior to application of irradiation or an anticancer drug, and the tumor sample is tumor cells after application of irradiation or an anticancer drug. Preferably, the control tumor cell sample is tumor cells prior to application of irradiation, and the tumor sample is tumor cells 3 to 5 days after application of irradiation. Preferably, the control tumor cell sample is tumor cells prior to application of an anticancer drug and the tumor sample is tumor cells 3 to 5 days after application of an anticancer drug. By using the tumor cells of the control tumor cell sample and the tumor sample before and after application of irradiation or anticancer drugs, it is possible to determine the senescence degree of tumor cells in response to treatments of irradiation or anticancer drugs on tumor cells. Accordingly, the aforesaid method enables determination of disease prognosis in response to anticancer therapy. Using the tumor cells taken 3 to 5 days after application of irradiation or anticancer drugs to the tumor sample, it is possible to determine changes of protein levels in a state which provides good detection sensitivity due to a sufficient progress of irradiation-induced senescence or anticancer drug-induced senescence of tumor cells.

A specific example of tumor cells is one or more selected from breast cancer cells, lung cancer cells and colon cancer cells.

The term “premature senescence” means that tumor cells become senescent by application of irradiation and/or anticancer drugs. A specific example of the anticancer drug is camptothecin, etoposide, and/or doxorubicin.

In another aspect, the present invention provides a method for detecting a premature senescence state of tumor cells in a human subject, comprising: (a) detecting a level of a target protein in a tumor sample obtained from the human subject, wherein the protein is one or more selected from the group consisting of eukaryotic elongation factor 1 alpha 1 (eEF1A1) and cathepsin D (CD); and (b) comparing a level of the target protein of Step (a) and a level of the same protein in a control tumor cell sample to determine one or more of decrease of eEF1A1 level and increase of CD level in the tumor sample.

In yet another aspect, the present invention provides a method for the diagnosis or prognosis of a premalignant or malignant state of tumor cells in a human subject, comprising: (a) detecting a level of a target protein in a tumor sample obtained from the human subject, wherein the protein is one or more selected from the group consisting of eukaryotic elongation factor 1 alpha 1 (eEF1A1 ) and cathepsin D (CD); and (b) comparing a level of the target protein of Step (a) and a level of the same protein in a control tumor cell sample to determine one or more of decrease of eEF1A1 level and increase of CD level in the tumor sample.

Since it is known that the characteristic phenotypes of premature senescence are abundant in a premalignant state of tumor cells, unlike malignant state, it is possible to make a diagnosis or prognosis of a premalignant or malignant state of tumor cells in a human subject by using the aforesaid method.

In yet another aspect, the present invention provides a method of screening for a premature senescence-inducing agent in a tumor cell, comprising: (a) collecting a first tumor cell sample before exposing to a candidate premature senescence-inducing agent; (b) collecting a second tumor cell sample after exposing to a candidate premature senescence-inducing agent; (c) detecting levels of one or more of CD and eEF1A1 in the first and second samples; (d) determining whether a level of CD increases and/or whether a level of eEF1A1 decreases; and (e) identifying a candidate agent exhibiting one or more of an increased CD level and a decreased eEF1A1 level, as a premature senescence-inducing agent.

In the step (c), using the second sample taken 3 to 5 days after exposing to the candidate agent, it is possible to determine changes of protein levels in a state which provides good detection sensitivity due to a sufficient progress of irradiation-induced senescence or anticancer drug-induced senescence of tumor cells.

Since an increased level of CD and/or a decreased level of eEF1A1 are specific to premature senescence of tumor cells, it is possible to screen premature senescence-inducing agents by using the aforesaid method. In addition, the method of the present invention may be used for screening potential candidates of cancer therapy, since an increased level of CD and/or a decreased level of eEF1A1 are specific to premature senescence of tumor cells.

In yet another aspect, the present invention provides a method of screening a potential candidate of cancer therapy in a human subject, the method comprising: (a) collecting a first tumor cell sample before exposing to a candidate premature senescence-inducing agent; (b) collecting a second tumor cell sample after exposing to a candidate premature senescence-inducing agent; (c) detecting levels of one or more of CD and eEF1A1 in the first and second samples; (d) determining whether a level of CD increases and/or whether a level of eEF1A1 decreases; and (e) identifying a potential cancer therapy agent exhibiting one or more of an increased CD level and a decreased eEF1A1 level, as a premature senescence-inducing agent.

In the step (c), using the second sample taken 3 to 5 days after exposing to the candidate agent, it is possible to determine changes of protein levels in a state which provides good detection sensitivity due to a sufficient progress of irradiation-induced senescence or anticancer drug-induced senescence of tumor cells.

Details and embodiments disclosed in connection with the method for detecting premature senescence in tumor cells will equally apply to the method for detecting a premature senescence state of tumor cells in a human subject, the method for the diagnosis or prognosis of a premalignant or malignant state of tumor cells in a human subject, the method of screening for a premature senescence-inducing agent in a tumor cell, and the method of screening a potential candidate of cancer therapy in a human subject, so long as there is no contradiction between them.

Further, the present invention provides a kit for detecting premature senescence in tumor cells, including antibodies directed against eEF1A1 and antibodies directed against CD.

The eEF1A1 - or CD-directed antibodies that can be used in the present invention may be monoclonal antibodies constructed according to any conventional production method of monoclonal antibodies known in the art or otherwise may be commercially available. The monoclonal antibodies may be quantitatively analyzed typically by color development using secondary antibodies conjugated with an enzyme such as alkaline phosphatase(AP) or horseradish peroxidase(HRP) and corresponding substrates thereof or otherwise may be quantitatively analyzed by directly using AP or HRP-conjugated monoclonal antibodies. In addition, polyclonal antibodies capable of recognizing eEF1A1 or CD may also be used instead of monoclonal antibodies. The polyclonal antibodies may be constructed according to any conventional production method of antisera known in the art.

The detection kit may be fabricated by any conventional method known in the art (see, e.g., D. Wild. 1994. The Immunoassay Handbook, 1st ed. Stockton Press, New York, USA, pp.89-91).

Further, the kit for detecting premature senescence in tumor cells may include a material for understanding the reaction between the antibodies and the tumor sample, for example a material for application of a protein chip or the like, in addition to antibodies.

Further, the detection kit including the antibodies of the present invention may contain antibodies and buffer, a stabilizer, inactive proteins and the like, typically in the freeze-dried form.

The antibodies may be labeled with radionuclides, fluorescors, enzymes or the like.

The CD protein may be encoded by a CD gene having a base sequence of SEQ ID NO: 1 (NCBI Access No. BC016320) or having a base sequence with deletion, substitution or insertion of one or more bases.

The eEF1A1 protein may be encoded by an eEF1A1 gene having a base sequence of SEQ ID NO: 2 (NCBI Access No. BC071619) or having a base sequence with deletion, substitution or insertion of one or more bases.

It will be apparent to those skilled in the art that above-illustrated sequences of the CD gene and eEF1A1 gene are provided for illustrative purposes only and the present invention is not limited thereto. Sequence derivatives or analogues having substantial sequence identity or substantial sequence homology with the above-illustrated sequences also fall within the scope of the present invention. As used herein, the term “substantial sequence identity” or “substantial sequence homology” is intended to indicate that a sequence exhibits substantial structural or functional equivalence with another sequence. Differences may also be due to inherent variations in codon usage among different species. Structural differences are considered de minimis if there is a significant amount of sequence overlap or similarity between two or more different sequences or if the different sequences exhibit similar physical characteristics even if the sequences differ in length or structure.

Details relating to genetic engineering techniques in the present invention can be found in the following literature: Sambrook, et al. Molecular Cloning, A Laboratory Manual, Cold Spring Harbor laboratory Press, Cold Spring Harbor, N.Y. (2001); and Frederick M. Ausubel et al., Current protocols in molecular biology volume 1, 2, 3, John Wiley & Sons, Inc. (1994).

According to the method and kit of the present invention, cellular senescence and senescence degree of tumor cells can be detected. Moreover, the discrimination between premature senescence and other cell fates (apoptosis or autophagy) of tumor cells can be made by detecting decrease of eEF1A1 level and increase of CD level in tumor cells, using the method and kit of the present invention. That is, apoptosis of tumor cells can be determined by detecting no changes in a level of eEF1A1 and decrease of CD level. Transient cell cycle arrest of tumor cells can be determined by detecting no changes in levels of eEF1A1 and CD. In addition, autophagy of tumor cells can be determined by detecting decrease of eEF1A1 and CD levels. As a consequence, it is possible to distinguish between premature senescence of tumor cells and other cell fates by detecting decrease of eEF1Al level and increase of CD level.

The following examples are provided to demonstrate and further illustrate certain preferred embodiments and aspects of the present invention and are not to be construed as limiting the scope thereof In the experimental disclosure which follows, the following abbreviations apply; min(minute); D or d (day or days); eq (equivalents); M (Molar); μM (micromolar); N (normal); mol (moles); mmol (millimoles); μmol (micromoles); nmol (nanomoles); g (grams); mg (milligrams); μg (micrograms); ng (nanograms); 1 or L (liters); mL (milliliters); μl (microliters); cm (centimeters); mm (millimeters); μm (micrometers); nm (nanometers); (degrees Centigrade); rpm(revolutions per minute); SDS (sodium dodecyl sulfate); FBS (fetal bovine serum); DMEM (Dulbecco's modified Earle's medium); IR (irradiation); CHAPS ([(3-cholamidopropyl)-dimethylammonio]-1-propane-sulfonate); PBS (phosphate buffered saline); DTT (dithiothreitol); PMSF (phenylmethanesulphonyl fluoride); IPG (immobilized pH gradient); TFA (trifluoroacetic acid); MALDI-TOF (Matrix-Assisted Laser Desorption Ionization time-of-flight); ATCC (American Type Culture Collection); RPMI (Rosewell Park Memorial Institute); NCBI (National Center for Biotechnology Information); Doxo (doxorubicin); Con (control); PARP (Poly(ADP-ribose) polymerase); ADP (adenosine diphosphate); DTB (double thymidine block); TAM (tamoxifen); CPT (camptothecin); ETS (etoposide); SA-β-gal (senescence-associated β-galactosidase); P-pRb (phosphorylated retinoblastoma); CDK2 (cyclin-dependent kinase 2); and PAGE (polyacrylamide gel electrophoresis). Although any methods and materials similar or equivalent to those described herein can be used in the practice or testing of the present invention, preferred methods and materials are described.

### EXAMPLE 1

### Detection of Irradiation-Induced Senescence in Breast Cancer Cell Line

1-1. Cell Culture and Treatments

The breast cancer cell line MCF-7 (ATCC, USA) as a tumor cell line was cultured in a medium supplemented with 10% fetal bovine serum (FBS, WelGENE, Daegu, Korea), 100 μg/mL of streptomycin and 100 units/mL of penicillin (Gibco BRL, USA) in a 5% CO₂ incubator at 37. The culture medium was DMEM (WelGENE, Daegu, Korea).

For irradiation, the breast cancer cell line was exposed to 6 Gy IR with a ¹³⁷Cs gamma-ray source (Atomic Energy of Canada Ltd., Mississauga, Ontario, Canada) at a dose rate of 3.81 Gy/min. Then, the cells were cultured in a 5% CO₂ incubator at 37 for 1 to 4 days.

Out of the breast cancer cell line, the cell line not exposed to gamma-ray irradiation was used as a control group.

1-2. Confirmation of Irradiation-Induced Senescence

In order to confirm premature senescence, characteristic morphological changes of cells in the control group and the breast cancer cell line 4 days after irradiation exposure thereof, each of which prepared in Section 1-1, were examined under a microscope (ECLIPSE TE300, Nikon).

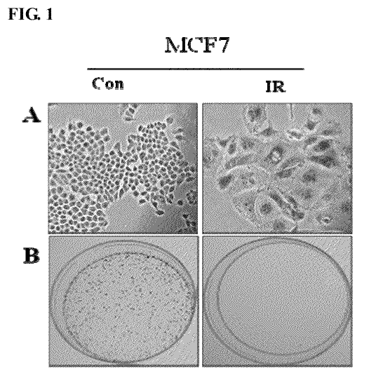

The results obtained are given in FIG. 1A. FIG. 1A shows a photograph of the MCF7 cell line taken on Day 4 after irradiation exposure of thereof (IR) and a photograph of the control group (Con). As confirmed from the results of FIG. 1A, the breast cancer cell line 4 days after irradiation exposure exhibited characteristics of senescent cells such as increased cell size and flat cell shape, unlike the control group.

1-3. Confirmation of Irradiation-Induced Senescence of Breast Cancer Cell Line by Colony Formation Assay

The control group and the irradiation-exposed breast cancer cell line (MCF7) of Section 1-1 were sub-cultured such that 5×10² cells were distributed on a 60 mm culture dish. Cell colonies formed after 7 to 10 day-culture of the cells in a cell incubator were stained with a Diff-Quick reagent (Sysmex Cat #38721). First, the medium was removed, and the cells were washed once with PBS, gently mixed with 0.5 mL of Solution A, followed by removal of cells, and then gently mixed with 0.5 mL of Solution B, followed by removal of cells. 0.5 mL of Solution C was added thereto, followed by gentle mixing and removal of cells. The cells were washed with a sufficient amount of distilled water, dried at room temperature for 30 min, and then subjected to colony formation assay using a colony counter (Imaging Products International #880). FIG. 1B is a photograph showing the results of colony formation assay, for the control group (Con) and the MCF7 cell line 7 days after irradiation exposure. As can be seen from the results of FIG. 1B, the irradiated senescent breast cancer cell line exhibited no colony formation.

Therefore, it was confirmed that proliferation of the tumor cell line was inhibited by irradiation-induced senescence.

1-4. Confirmation of Irradiation-Induced Senescence of Breast Cancer Cell Line by Senescence-Associated β-Galactosidase Activity Staining

In order to confirm irradiation-induced senescence of a tumor cell line, senescence-associated β-galactosidase activity staining was conducted on the control group and the breast cancer cell line 1, 2, 3 and 4 days after irradiation exposure, each of which prepared in Section 1-1. The cell staining was carried out according to a known method as described in Dimri et al., Proc. Natl. Acad. Sci. USA, 92:9363-9367, 1995.

The cells were washed twice with PBS and fixed with 3% formaldehyde at room temperature for 3 to 5 min. The fixed cells were washed once again with PBS, and 5 mL of a β-galactosidase activity staining solution (1 mg/mL X-Gal, 40 mM citric acid/sodium phosphate (pH 6.0), 5 mM potassium ferrocyanide, 5 mM potassium ferricyanide, 150 mM sodium chloride, and 2 mM magnesium chloride) was added thereto, followed by reaction in an incubator at 37 for 12 to 16 hours. During the reaction, the cells were cultured under light-shielding conditions by wrapping of the culture dish in silver foil.

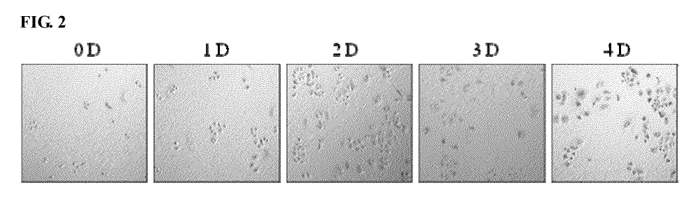

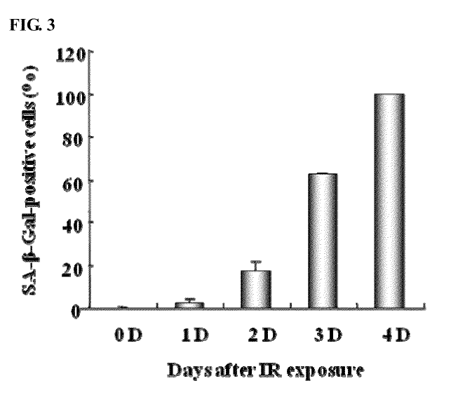

β-galactosidase activity was examined under a phase contrast microscope (ECLIPSE TE300, Nikon). The results obtained are shown in FIG. 2. FIG. 2 shows the results of senescence-associated β-galactosidase activity staining of the breast cancer cell line 1, 2, 3 and 4 days after irradiation exposure. As a result, premature senescence gradually proceeds over time.

Further, the stained cells were counted under a microscope (ECLIPSE TE300, Nikon) following irradiation exposure of the cells. The results obtained are shown in FIG. 3. FIG. 3 is a graph showing a time-varying portion (%) of SA-β-Gal-positive cells after irradiation exposure of the cell line. As a consequence, it was confirmed that the number of the stained cells was gradually increased, and almost all the remaining cells exhibited the β-galactosidase activity 4 days after irradiation exposure. That is, it was demonstrated from the above results that irradiation-induced premature senescence of the cancer cell line was evidently progressed when 4 days have passed after irradiation exposure of the cell line.

1-5. Confirmation of Irradiation-Induced Senescence Biomarkers

(1) Preparation of Samples

The breast cancer cell line (MCF7) of Section 1-1, taken 4 days after irradiation exposure was applied to the cells, was washed with PBS, and the cell sample was subjected to protein extraction using cell lysis buffer {9M urea, 2M thiourea, 100 mM DTT, 2% CHAPS (w/v), 60 mM n-octyl-D-glucopyranoside, 2% IPG buffer (pH 3-10, Amersham Biosciences, Piscataway, N.J.), and 1 mM PMSF} for two-dimensional electrophoresis. The extracted protein was centrifuged at 14,000 rpm for 10 min. The supernatant was collected and subjected to quantitative analysis using Bradford protein assay {Bradford, M., Anal. Biochem. 72:248-254 (1976)}. Firstly, three to five dilutions of a protein standard, which is representative of the protein solution to be tested were prepared. Next, 100 μl of each standard and diluted sample solution was taken into cuvets and 0.9 mL of diluted dye reagent was added to each cuvet. Mixtures were vortexed and then incubated at room temperature for at least 5 minutes. Finally, absorbance of samples was measured at 595 nm with a UV spectrophotometer (Amersham Biosciences, USA, Ultrospec 3100, #80-2112-31).

(2) Two-Dimensional Electrophoresis

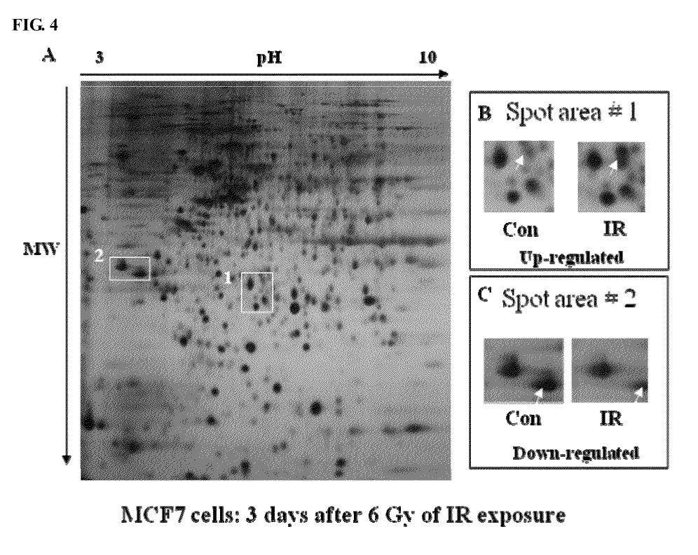

According to Bradford protein assay, a rehydration solution (8M urea, 2% CHAPS, 0.5% IPG buffer, and 0.002% bromophenol blue) was added to the control group and 120 μg of the quantified protein, followed by sufficient mixing. Each mixture was placed on an IPG (immobilized pH gradient: pH 3-10, length: 24 cm) strip and subjected to one-dimensional isoelectric focusing electrophoresis at 20 for 81,780 Vhr using an IPGphor system (Amersham Biosciences, Piscataway, N.J.). Two-dimensional electrophoresis was performed on SDS-PAGE gel (11% polyacrylamide, 0.26% 1,4-bis(acryloyl)piperazine (PDA)/25.5 cm×19.6 cm×1 mm) at a voltage of 2 W, using an Ettandalt 6 electrophoresis system (Amersham Biosciences). The gel images were collected using a silver staining kit (Amersham Biosciences). Representative gel images are shown in FIG. 4A. FIG. 4A shows two-dimensional protein electrophoretic patterns for the comparison of protein bodies. FIG. 4B is an enlarged view of an area having an increased level (Spot area #1) as compared to the control group, and FIG. 4C is an enlarged view of an area having a decreased level (Spot area #2) as compared to the control group.

(3) Mass Analysis

With reference to the gel images of 4A, two protein spots (FIGS. 4B and 4C), which exhibited differential level in the non-irradiated control tumor cell group having cell proliferative capacity and the irradiated senescent cell group with loss of cell proliferative capacity, were excised and subjected to in-gel trypsin digestion (Shevehenko et al., Rapid Commun Mass Spectrom. 11:1015-1024, 1997). The excised gel spots were then destained and incubated using 200 mM ammonium bicarbonate for 20 min. These gel pieces were dehydrated and dried, and then rehydrated. The peptide solution was desalted with a C₁₈ nano column (IN2GEN Co., Ltd., Seoul, Korea). The MS/MS of peptides generated via in-gel digestion was conducted by nano-ESI on a Q-TOF2 mass spectrometer (Micromass, Manchester, UK). The productions were analyzed with an orthogonal TOF analyzer. The data were processed with a Mass Lynx Windows NTPC system.

The digested protein spots were confirmed by MALDI-TOF. The MALDI-TOF analysis was performed using a delayed-extraction reflectron time-of-flight mass spectrometer (Model M@LDI-R; Micromass. Manchester, UK) with an accelerating voltage of 20 kV and a grid voltage of 65%. The MALDI-TOF analysis employed 0.5 μl of a matrix solution containing 10 mg/mL α-cyano-4-hydroxycinnamic acid (CHCA), 0.1% TFA, and 50% acetonitrile and 5 μl of the trypsin-digested amino acid solution, and was automatically carried out by a MALDI-TOF analyzer. Based on the mass spectrometric results of peptides obtained, identification of peptides was performed in the NCBInr database, using a MASCOT program (www.matrixscience.com).

When two-dimensional electrophoresis and mass analysis were performed as above, certain proteins, which show altered levels in irradiation-induced senescent cells, were identified as follows (Table 1).

TABLE 1

| Spot | | NCBI Access | | |
|---|---|---|---|---|
| No. | Protein | No. | Mass | Mascot score |
| 1 | Eukaryotic translation | BC067787 | 24919 | 98 |
| | elongation factor 1 beta 2 | | | |
| 2 | Chain B, Cathepsin D | BC016320 | 26457 | 60 |

CD (Cathepsin D) was selected as an irradiation-induced senescence biomarker. In addition, eEF1A1 (eukaryotic translation elongation factor 1 alpha 1; NCBI Access No. BC071619) was also selected as an irradiation-induced senescence biomarker, based on the fact that eEF1B2 (eukaryotic translation elongation factor 1 beta 2) participates in the control of an activity of eEF1A1 (eukaryotic translation elongation factor 1 alpha 1) through the formation of a complex with eEF1A1 during intracellular protein synthesis.

With methods of detecting levels of the above proteins using protein-directed antibodies, irradiation-induced premature senescence of the tumor cell line can be detected.

1-6. Detection of Irradiation-Induced Senescence by Western Blot Analysis

The breast cancer cell line (MCF7) of Section 1-1, taken 4 days after irradiation exposure was applied to the cells, was washed with PBS, and the cell sample was subjected to protein extraction using a cell lysis buffer (50 mM Tri-HCl, 1% NP-40, 0.25% sodium deoxycholate, 150 mM NaCl, 1 mM PMSF, 50 mM NaF, 0.2 mM Na₃VO₄, 10 g/mL aprotinin, and 2 g/mL leupeptin). The extracted protein was centrifuged at 11,000 rpm for 10 min. The supernatant was collected and subjected to quantitative analysis using Bradford protein assay (Bradford, M., Anal. Biochem. 72:248-254 (1976)). 20 μg of the protein was added to 2× SDS loading buffer (60 mm Tris-Cl (pH 6.8), 25% glycerol, 2% SDS, 14.4 mM mercaptoethanol, and 0.1% bromophenol blue), and the mixture was heated at 95 for 5 min and subjected to electrophoresis on 8% SDS polyacrylamide gel at 80V for 2 hours. After the electrophoresis was complete, the separated protein was transferred to a nitrocellulose membrane (Whatman). The protein-transferred membrane was placed in a PBS solution containing 5% nonfat dry milk, and allowed to stand at room temperature for 1 hour to be blocked. Primary antibodies diluted to 1:1000 were added thereto, followed by reaction at 4 for 16 hours. The primary antibodies were polyclonal anti-eEF1A1 antibodies (Abcam, Mass., USA, Cat # ab37969 ) and anti-CD antibodies (Santa Cruz Biotechnology, Calif., USA, Cat # SC-6494 ). The secondary antibodies were horseradish peroxidase-conjugated anti-rabbit antibodies (GE healthcare, Backinghanshire, UK, Cat # NA934V). In addition, an enhanced chemiluminescence (ECL) reagent (Amersham Biosciences, Piscataway, N.J.) was used for confirmation of the reaction. Actin was used as an internal positive control, and primary antibodies were anti-actin antibodies (Santa Cruz Biotechnology, Calif., USA, Cat # SC-1616).

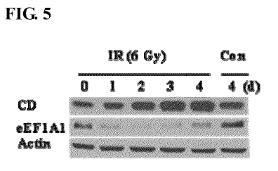

The results obtained are shown in FIG. 5. FIG. 5 shows Western blotting results to confirm changes in levels of CD and eEF1A1 in the irradiation-induced senescent cell line.

As a result, a gradual quantitative decrease of eEF1A1 and a gradual quantitative increase of CD were observed in response to irradiation-induced premature senescence. Dramatic changes in levels of eEF1A1 and CD were confirmed in the control group and on Day 4 at which treatment-induced senescence was complete.

The intensity of bands according to Western blot analysis was measured and compared by image scanning analysis as follows.

The signal intensity was scanned with HP Scanjet 2400 (Hewlett-Packard Company, Palo Alto, Calif., USA) and analyzed with ImageJ Program (http://rsb.info.nih.gov/ij/). When quantitative changes of the proteins were measured, CD and eEF1A1 exhibited values of 2.25 and 0.52, respectively, on Day 4 provided that Day 0=1.

Therefore, it can be undoubtedly seen that eEF1A1 and CD are biomarkers for irradiation-induced senescence of breast cancer cells. In addition, it can also be seen that irradiation-induced senescence of tumor cell lines is detectable using antibodies directed against eEF1A1 and/or CD. With detection of eEF1A1 and CD levels according to Western blot analysis using the eEF1A1 and/or CD-directed antibodies, the irradiation-induced senescence degree of tumor cell lines can be determined by naked eyes directly through the band intensity on the gel.

Upon analysis of the signal intensity using image scanning analysis, the degree of premature senescence can be quantitatively determined.

### EXAMPLE 2

### Detection of Irradiation-Induced Senescence in Lung Cancer Cell Line

2-1. Cell Culture and Treatments

The irradiation-exposed cell line and control group were prepared in the same manner as in Section 1-1 of Example 1, except that the lung cancer cell line H460 (ATCC, USA) was used as a tumor cell line, and RPMI 1640 (WelGENE, Daegu, Korea) was used as a culture medium.

2-2. Confirmation of Irradiation-Induced Senescence of Lung Cancer Cell Line by Senescence-associated β-Galactosidase Activity Staining

In order to confirm irradiation-induced senescence of a tumor cell line, the procedure was performed in the same manner as in Section 1-4 of Example 1, except that the control group and the lung cancer cell line 4 days after irradiation exposure thereof, each of which prepared in Section 2-1, were used.

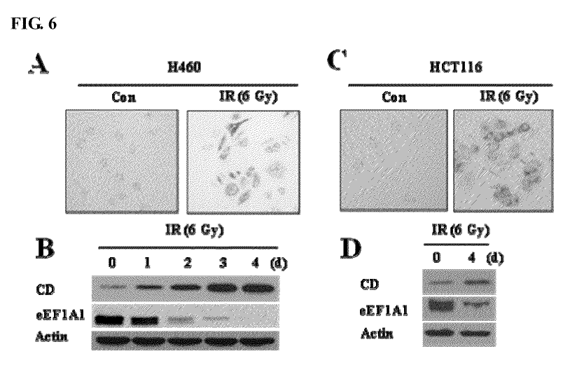

β-galactosidase activity was examined under a phase contrast microscope (ECLIPSE TE300, Nikon). The results obtained are shown in FIG. 6A. FIG. 6A is a staining picture of the senescence-associated β-galactosidase activity in response to irradiation exposure of the H460 cell line. From the results of FIG. 6A, it was confirmed that counts of stained cells were greatly increased 4 days later, as compared to the control group. That is, the non-irradiated lung cancer cell line exhibited no premature senescence, but underwent significant premature senescence 4 days after the cells were exposed to irradiation.

From these results, it was confirmed that premature senescence of the lung cancer cell line progressed to a significant level, after the passage of 4 days following irradiation exposure of the cell line.

Accordingly, it can be seen that the detection of irradiation-induced tumor cell senescence can have high detection sensitivity when 4 days have passed following irradiation exposure of the cell line.

2-3. Detection of Irradiation-Induced Senescence of Lung Cancer Cell Line by Western Blot Analysis

The procedure was performed in the same manner as in Section 1-6 of Example 1, except that the lung cancer cell line (H460) prepared in Section 2-1 and passed 1, 2, 3 and 4 days after application of irradiation to the cells and the lung cancer cell line prior to irradiation exposure were used.

The results obtained are shown in FIG. 6B. FIG. 6B shows Western blotting results confirming time-course changes (1, 2, 3, 4 days) in levels of eEF1A1 and CD in the H460 cell line in response to irradiation exposure thereof

As a result, a gradual quantitative decrease of eEF1A1 and a gradual quantitative increase of CD were observed in response to irradiation-induced premature senescence, and dramatic changes in levels of eEF1A1 and CD were confirmed on Day 4 at which treatment-induced senescence was complete.

The signal intensity was scanned with HP Scanjet 2400 (Hewlett-Packard Company, Palo Alto, Calif., USA) and analyzed with ImageJ Program (http://rsb.info.nih.gov/ij/). When quantitative changes of the proteins were measured, CD and eEF1A1 exhibited values of 10.43 and 0.0 1, respectively, on Day 4 provided that Day 0=1.

Therefore, it can be undoubtedly seen that eEF1A1 and CD are biomarkers for irradiation-induced senescence of lung cancer cells. In addition, it can also be seen that irradiation-induced senescence of tumor cell lines is detectable using antibodies directed against eEF1A1 and/or CD. With detection of eEF1A1 and CD levels according to Western blot analysis using the eEF1A1 and/or CD-directed antibodies, the irradiation-induced senescence degree of tumor cell lines can be determined by naked eyes directly through the band intensity on the gel. Upon analysis of the signal intensity using image scanning analysis, the degree of premature senescence can be quantitatively determined.

### EXAMPLE 3

### Detection of Irradiation-Induced Senescence in Colon Cancer Cell Line

3-1. Cell Culture and Treatments

Irradiation-exposed cell line and control group were prepared in the same manner as in Section 1-1 of Example 1, except that the colon cancer cell line HCT 116 (ATCC, USA) was used as a tumor cell line, and McCoy's 5A (WelGENE, Daegu, Korea) was used as a culture medium.

3-2. Confirmation of Irradiation-Induced Senescence of Colon Cancer Cell Line by Senescence-Associated β-Galactosidase Activity Staining

In order to confirm irradiation-induced senescence of a tumor cell line, the procedure was performed in the same manner as in Section 1-4 of Example 1, except that the control group and the colon cancer cell line (HCT 116) 4 days after irradiation exposure thereof, each of which prepared in Section 3-1, were used.

β-galactosidase activity was examined under a phase contrast microscope (ECLIPSE TE300, Nikon). The results obtained are shown in FIG. 6C. FIG. 6C is a staining picture of the senescence-associated P-galactosidase activity in response to irradiation exposure of the HCT116 cell line. From the results of FIG. 6C, it was confirmed that counts of stained cells were greatly increased 4 days later, as compared to the control group. That is, the non-irradiated colon cancer cell line exhibited no premature senescence, but underwent significant premature senescence 4 days after the cells were exposed to irradiation.

From these results, it was confirmed that premature senescence of the colon cancer cell line progressed to a significant level, after the passage of 4 days following irradiation exposure of the cell line.

Accordingly, it can be seen that the detection of irradiation-induced premature senescence can have high detection sensitivity when 4 days have passed following irradiation exposure of the cell line.

3-3. Detection of Irradiation-Induced Senescence of Lung Cancer Cell Line by Western Blot Analysis

The procedure was performed in the same manner as in Section 1-6 of Example 1, except that the colon cancer cell line (HCT116) prepared in Section 3-1 and passed 4 days after application of irradiation to the cells was used.

The results obtained are shown in FIG. 6D. FIG. 6D shows Western blotting results confirming time-course changes (4 days) in levels of eEF1A1 and CD in the HCT116 cell line in response to irradiation exposure. As a result, a quantitative decrease of eEF1A1 and a quantitative increase of CD were observed in response to irradiation-induced premature senescence.

The signal intensity was scanned with HP Scanjet 2400 (Hewlett-Packard Company, Palo Alto, Calif., USA) and analyzed with ImageJ Program (http://rsb.info.nih.gov/ij/). When quantitative changes of the proteins were measured, CD and eEF1A1 exhibited values of 2.98 and 0.44, respectively, on Day 4 provided that Day 0=1.

Therefore, it can be undoubtedly seen that eEF1A1 and CD are biomarkers for irradiation-induced senescence of colon cancer cells. In addition, it can also be seen that irradiation-induced senescence of tumor cell lines is detectable using antibodies directed against eEF1A1 and/or CD. With detection of eEF1A1 and CD levels according to Western blot analysis using the eEF1A1 and/or CD-directed antibodies, the irradiation-induced senescence degree of tumor cell lines can be determined by naked eyes directly through the band intensity on the gel. Upon analysis of the signal intensity using image scanning analysis, the degree of premature senescence of tumor cell can be quantitatively determined.

### EXAMPLE 4

### Detection of Doxorubicin-Induced Senescence in Breast Cancer Cell Line

Low doses of a chemotherapeutic drug, doxorubicin, induce premature senescence in carcinoma cells. In contrast, high doses of this drug induce apoptosis. In order to examine whether an increasing level of CD and a decreasing level of eEF1A1 are independent of apoptosis and are specific to premature senescence of tumor cells, experiments were carried out as follows.

4-1. Cell Culture and Treatments

Analogously to the procedure of Section 1-1 of Example 1, the cultured cell line (MCF7) was prepared.

The cell line was divided into three groups: a group with treatment of high-dose doxorubicin (10 μg/mL), a group with treatment of low-dose doxorubicin (50 ng/mL), and a group with no treatment of doxorubicin. The non-doxorubicin treated group was used as a control group.

4-2. Confirmation of Doxorubicin-Induced Senescence

In order to ascertain doxorubicin-induced senescence or apoptosis of a tumor cell line, the procedure was carried out in the same manner as in Section 1-2 of Example 1, except that the experiment was conducted according to a time-course schedule (0, 16, 24, 48, and 96 hours) using the cell line of Section 4-1.

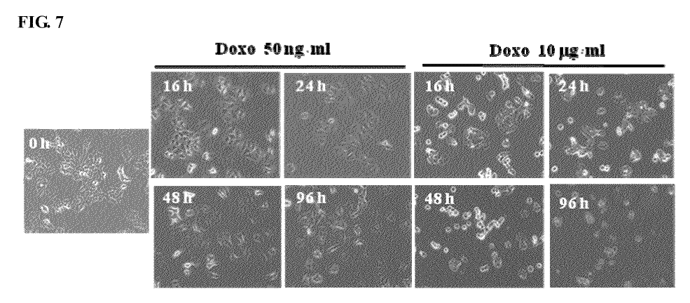

The results obtained are shown in FIG. 7. FIG. 7 shows micrographs of cell morphology over time in response to treatments of doxorubicin at a high dose (10 μg/mL) and a low dose (50 ng/mL), respectively. At a low dose, doxorubicin induced senescence-specific morphological changes. In contrast, a high dose of doxorubicin induced apoptosis-specific morphological changes.

Accordingly, it was demonstrated that low doses of doxorubicin induce premature senescence, whereas high doses of doxorubicin induce apoptosis.

4-3. Confirmation of Doxorubicin-Induced Senescence of Breast Cancer Cell Line by Senescence-Associated β-Galactosidase Activity Staining

In order to investigate low-dose doxorubicin-induced senescence of a tumor cell line, the procedure was performed in the same manner as in Section 1-3 of Example 1, except that the cell line prepared in Section 4-1 was used.

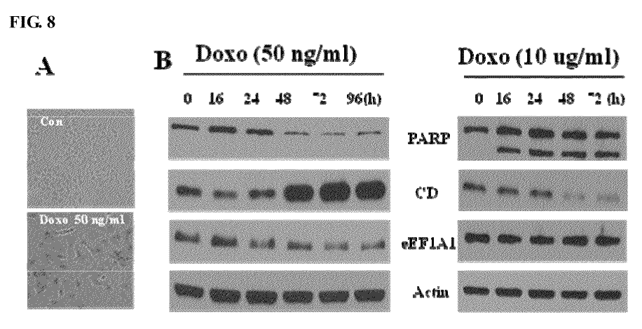

β-galactosidase activity was examined under a phase contrast microscope (ECLIPSE TE300, Nikon). The results obtained are shown in FIG. 8A. FIG. 8A shows the observation results for the cells 4 days after treatment of doxorubicin (50 ng/mL). At a low dose, doxorubicin induced positive SA-β-gal staining.

From these results, it was demonstrated that low doses of doxorubicin induce premature senescence.

4-4. Detection of Doxorubicin-Induced Senescence of Breast Cancer Cell Line by Western Blot Analysis

The procedure was performed in the same manner as in Section 1-6 of Example 1, except that the cell line with treatment of doxorubicin at a high dose and the cell line with treatment of doxorubicin at a low dose, each of which prepared in Section 4-1, were used. In order to confirm the occurrence of apoptosis, a level of poly(ADP-ribose) polymerase (PARP) serving as an apoptosis biomarker was detected according to Western blot analysis as in Section 1-6 of Example 1, except that polyclonal anti-PARP antibodies (Cell Signaling Technology, Mass., USA, Cat #9541) were used as primary antibodies.

The results obtained are shown in FIG. 8B. FIG. 8B shows Western blotting results confirming changes in levels of eEF1A1 and CD in the tumor cell line in response to treatment of high-dose doxorubicin (10 μg/mL) and treatment of low-dose doxorubicin (50 ng/mL).

When premature senescence was induced by low-dose treatment of doxorubicin, it was confirmed that a level of CD increases and a level of eEF1A1 decreases.

A high dose of doxorubicin (10 μg/mL) induced poly(ADP-ribose) polymerase (PARP) cleavage (apoptic marker). Under these apoptotic conditions, a protein level of eEF1A1 remained unaltered, whereas CD was gradually decreased.

Taken together, it can be seen that when premature senescence in tumor cells took place due to the treatment of doxorubicin, it is possible to detect premature senescence of tumor cells irrespective of apoptosis by detecting an increased level of CD and a decreased level of eEF1A1.

The signal intensity was scanned with HP Scanjet 2400 (Hewlett-Packard Company, Palo Alto, Calif., USA) and analyzed with ImageJ Program (http://rsb.info.nih.gov/ij/). When quantitative changes of the proteins were measured, CD and eEF1A1 exhibited values of 2.45 and 0.53, respectively, on Day 4 provided that Day 0=1.

Therefore, it can be seen that doxorubicin-induced senescence of tumor cell lines is detectable using antibodies directed against eEF1A1 and/or CD. With detection of eEF1A1 and CD levels according to Western blot analysis using the eEF1A1 and/or CD-directed antibodies, the doxorubicin-induced senescence degree of tumor cell lines can be determined by naked eyes directly through the band intensity on the gel. Upon analysis of the signal intensity using image scanning analysis, the degree of premature senescence can be quantitatively determined.

### EXAMPLE 5

### Detection of Doxorubicin-Induced Senescence in Lung Cancer Cell Line

Whether an increasing level of CD and a decreasing level of eEF1A1 are independent of apoptosis and are specific to premature senescence of tumor cells was confirmed according to the following method.

5-1. Cell Culture and Treatments

The cultured cell line was prepared in the same manner as in Section 1-1 of Example 1, except that the lung cancer cell line H460 (ATCC, USA) was used as a tumor cell line, and RPMI 1640 (WelGENE, Daegu, Korea) was used as a culture medium.

The cell line was treated analogously to the procedure as in Section 4-1 of Example 4.

5-2. Confirmation of Doxorubicin-Induced Senescence

In order to ascertain doxorubicin-induced senescence or apoptosis of a tumor cell line, the procedure was carried out in the same manner as in Section 1-2 of Example 1, except that the experiment was conducted 96 hours after treatment of doxorubicin on the cell line of Section 5-1.

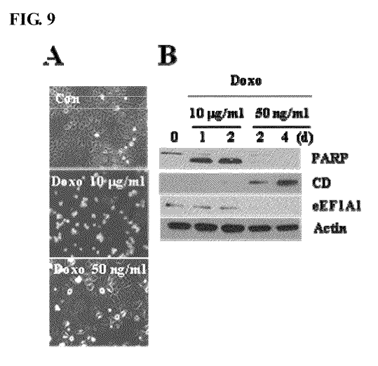

The results obtained are shown in FIG. 9A. FIG. 9A shows micrographs of cell morphology to confirm senescence or apoptosis of the tumor cell line in response to treatments of doxorubicin at a high dose (10 μg/mL) and a low dose (50 ng/mL), respectively. At a low dose, doxorubicin induced senescence-specific morphological changes. In contrast, a high dose of doxorubicin induced apoptosis-specific morphological changes.

Accordingly, it was demonstrated that low doses of doxorubicin induce cellular senescence, whereas high doses of doxorubicin induce apoptosis.

5-3. Detection of Doxorubicin-Induced Senescence of Lung Cancer Cell Line by Western Blot Analysis

The procedure was performed in the same manner as in Section 4-4 of Example 4, except that the cell line with treatment of doxorubicin at a high dose and the cell line with treatment of doxorubicin at a low dose, each of which prepared in Section 5-1, were used.

The results obtained are shown in FIG. 9B. FIG. 9B shows Western blotting results confirming changes in levels of eEF1A1 and CD in the tumor cell line in response to treatment of high-dose doxorubicin (10 μg/mL) and treatment of low-dose doxorubicin (50 ng/mL).

When premature senescence took place by low-dose treatment of doxorubicin, it was confirmed that a level of CD increases and a level of eEF1A1 decreases.

It was demonstrated that CD and eEF1A1 are biomarkers irrelevant to apoptosis and specific to premature senescence of tumor cells because there were no changes in levels of CD and eEF1A1 when apoptosis was induced by high-dose treatment of doxorubicin.

Therefore, it can be seen that doxorubicin-induced senescence of tumor cell lines is detectable using antibodies directed against eEF1A1 and/or CD. With detection of eEF1A1 and CD levels according to Western blot analysis using the eEF1A1 and/or CD-directed antibodies, the doxorubicin-induced senescence degree of tumor cell lines can be determined by naked eyes directly through the band intensity on the gel. Upon analysis of the signal intensity using image scanning analysis, the degree of premature senescence can be quantitatively determined.

### EXAMPLE 6

### Detection of Anticancer Drug-Induced Senescence in Breast Cancer Cell Line

6-1. Cell Culture and Treatments

Analogously to the procedure of Section 1-1 of Example 1, the same cell line was cultured and prepared.

The cell line was divided into two groups: a group with treatment of camptothecin (40 nM and a group with treatment of etoposide (30 μM). In addition, the non-treated cell line was divided into two groups which will serve as a control group for each drug-treated group.

Camptothecin and etoposide were purchased from Calbiochem (La Jolla, Calif., USA).

6-2. Confirmation of Anticancer Drug-Induced Senescence of Breast Cancer Cell Line by Senescence-Associated β-Galactosidase Activity Staining

In order to investigate anticancer drug-induced senescence of a tumor cell line, the procedure was performed in the same manner as in Section 1-4 of Example 1, except that the cell line prepared in Section 6-1 was used.

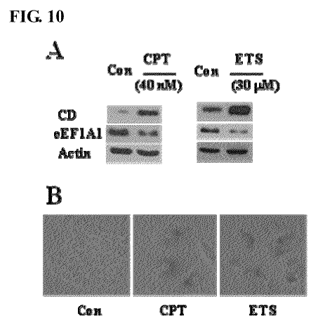

β-galactosidase activity was examined under a phase contrast microscope (ECLIPSE TE300, Nikon). The results obtained are shown in FIG. 10B. FIG. 10B shows a staining picture of the senescence-associated β-galactosidase activity upon treatments of the breast cancer cell line with camptothecin and etoposide.

As a result, both the camptothecin-treated group and the etoposide-treated group exhibited characteristics of premature senescence, unlike the control group.

From these results, it was demonstrated that both camptothecin and etoposide induce cellular senescence.

6-3. Detection of Anticancer Drug-Induced Senescence of Breast Cancer Cell Line by Western Blot Analysis

The procedure was performed in the same manner as in Section 1-6 of Example 1, except that the cell line prepared in Section 6-1 was used.

The results obtained are shown in FIG. 10A. FIG. 10A shows Western blotting results confirming changes in levels of eEF1A1 and CD in the breast cancer cell line in response to treatments of camptothesin and etoposide.

It was confirmed that a level of CD increases and a level of eEF1A1 decreases, when the cancer cell line was treated with camptothesin and etoposide.

The signal intensity was scanned with HP Scanjet 2400 (Hewlett-Packard Company, Palo Alto, Calif., USA) and analyzed with ImageJ Program (http://rsb.info.nih.gov/ij/). When quantitative changes of the proteins were measured, upon treatment of camptothecin, CD and eEF1A1 exhibited values of 7.46 and 0.73, respectively, provided that each control group (Con)=1. Upon treatment of etoposide, CD and eEF1A1 exhibited values of 2.24 and 0.25, respectively, provided that each control group (Con)=1.

From these results, it was demonstrated that eEF1A1 and/or CD are biomarkers specific to camptothecin- and etoposide-induced senescence.

Therefore, it can be seen that camptothecin and/or etoposide-induced senescence of tumor cell lines is detectable using antibodies directed against eEF1A1 and/or CD. With detection of eEF1A1 and CD levels according to Western blot analysis using the eEF1A1 and/or CD-directed antibodies, the anticancer drug-induced senescence degree of tumor cell lines can be determined by naked eyes directly through the band intensity on the gel. Upon analysis of the signal intensity using image scanning analysis, the degree of premature senescence can be quantitatively determined.

### EXAMPLE 7

### Demonstration of Whether a Decreasing Level of eEF1A1 and an Increasing Level of CD are Specific to Premature Senescence of Tumor Cells

This example is intended to demonstrate that a decreasing level of eEF1A1 and an increasing level of CD are specific to premature senescence and are not correlated with transient cell cycle arrest. For this purpose, the experiment was performed according to the following method.

Transient arrest at G1/S phase was induced by double thymidine block (DTB). (Haper J V. Synchronization of cell population in G1/S and G2/M phases of the cell cycle. Methods Mol Biol 2005; 296:157-66).

The same cell line was cultured and used in the same manner as in Section 1-1 of Example 1.

Cells were synchronized at G1/S by a double thymidine treatment (15 h, 0.5 mg/mL) separated by an 8 h interval (arrested). After synchronization, cells were released from DTB and incubated for additional 48 h (released). Control cells were maintained in an identical manner except that they were kept in media containing thymidine.

P-pRb and CDK2 were used as markers of G1/S phase transition arrest. Protein levels were confirmed by Western blot analysis in the same manner as in Section 1-6 of Example 1, except that primary antibodies directed against P-pRb were Phosphoro-pRb antibodies (Cell Signaling Technology, Danvers, Mass., Cat #9308), and primary antibodies directed against CDK2 were anti-CDK2 antibodies (Santa Cruze Biotechnology, Calif., USA, Cat #SC-163).

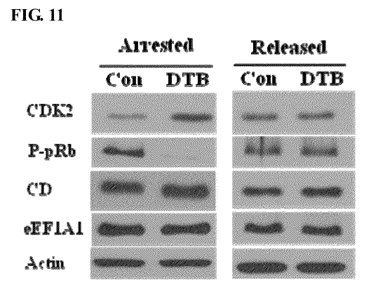

The results obtained are shown in FIG. 11. FIG. 11 shows Western blotting results for understanding the relationship between transient cell cycle arrest and level changes of eEF1A1 and CD.

G1/S phase transition arrest was evidenced by a decrease in phosphorylated pRb and an accumulation in CDK2. No significant alteration in protein levels of eEF1A1 and CD was detected (FIG. 11, left). Even after release from DTB, cells did not exhibit any alteration in eEF1A1 and CD levels.

Accordingly, it was demonstrated that a decreasing level of eEF1A1 and an increasing level of CD are specific to premature senescence of tumor cells and are independent of transient cell cycle arrest. Therefore, it is possible to selectively detect premature senescence of tumor cells by detecting changes in levels of eEF1A1 and/or CD, which consequently allows for cell fate prediction of tumor cells.

### EXAMPLE 8

### Demonstration of Whether Changes in Levels of eEF 1A1 and CD are Specific to Premature Senescence

Autophagy has been reported to occur in several types of cancer cells in response to radiation or chemotherapy (Kim E H, Sohn S, Kwon H J, et al. Sodium selenite induces superoxide-mediated mitochondrial damage and subsequent autophagic cell death in malignant glioma cells. Cancer Res 2007; 57:6314-24).

Therefore, the following method was performed to demonstrate that a decreasing level of eEF1A1 and an increasing level of CD are independent of autophagy and are specific to premature senescence of tumor cells.

8-1. Cell Culture and Treatments

The same cell line as in Section 1-1 of Example 1 was cultured and used in an identical method.

The cell line was divided into two groups: a group which was treated with tamoxifen (10 μmol/L) for 2 days, and a non-treated group as a control group.

Tamoxifen was purchased from Calbiochem (La Jolla, Calif., USA).

8-2. Confirmation of Tamoxifen-Induced Autophagy

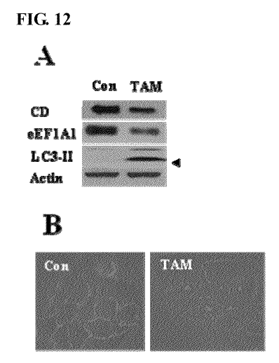

Morphological changes of cells in the control group and the tamoxifen-treated group of Section 8-1 were examined under a microscope (ECLIPSE TE300, Nikon). The results obtained are shown in FIG. 12B. FIG. 12B shows microscopic observations of cell morphology with tamoxifen-induced autophagy (B). Therefore, it was confirmed that the tamoxifen-treated group exhibited the occurrence of autophagy due to disintegration of intracellular organelles, unlike the control group.

8-3. Confirmation of Level Changes of CD and eEF1A1 by Western Blot Analysis

The procedure was performed in the same manner as in Section 1-6 of Example 1, except that the cell line prepared in Section 8-1 was used. In order to detect changes in levels of LC3-II which is an autophagy biomarker, anti-LC3-II antibodies (Sigma, Mo., USA, Cat #L8919) were used as primary antibodies directed against LC3-II.

The results obtained are shown in FIG. 12 A. FIG. 12A shows Western blotting results for understanding the relationship between autophagy and level changes of eEF1A1 and CD.

The tamoxifen-treated group exhibited an increased level of LC3-II, thereby confirming the occurrence of autophagy. Both CD and eEF1A1 were decreased under these conditions, showing that premature senescence can be distinguished from autophagy, based on CD and eEF1A1 levels.

These results show that CD and eEF1A1 are relevant markers for detecting a variety of types of premature senescence and that both markers are sufficient to distinguish premature senescence from other cellular fates (i.e., autophagy or apoptosis).

As can be seen from the results of non-limiting examples as illustrated above, the detection of levels of CD and eEF1A1 in tumor cells has a variety of applications including, for example, detecting a premature senescence state of tumor cells in a human subject, diagnosis or prognosis of a premalignant or malignant state of tumor cells in a human subject, screening for premature senescence-inducing agents in tumor cells, screening potential candidates of cancer therapy in a human subject, and the like.

### PREPARATION EXAMPLE 1

### Preparation of a Kit for Detecting Premature Senescence in Tumor Cells

Polyclonal anti-eEF1A1 antibodies (ab37969, Abcam) and anti-CD antibodies (SC-6469, Santa Cruz Biotechnology) were packaged into one container to thereby prepare a kit for the detection of premature senescence in tumor cells, containing eEF1A1 antibodies and CD antibodies. Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method for detecting premature senescence in tumor cells, comprising:
(a) detecting a level of a target protein in a tumor sample, wherein the protein is one or more selected from the group consisting of eukaryotic elongation factor 1 alpha 1 (eEF1A1) and cathepsin D (CD); and
(b) comparing a level of the target protein of Step (a) and a level of the same protein in a control tumor cell sample to determine one or more of decrease of eEF1A1 level and increase of CD level in the tumor sample.

2. The method according to claim 1, wherein the tumor sample is tumor cells or tumor tissues.

3. The method according to claim 2, wherein the cells or the tissues are derived from a mammal.

4. The method according to claim 1, wherein the control tumor cell sample is non-senescent cells.

5. The method according to claim 1, wherein the detection of a protein level is by antibodies that specifically bind to the protein.

6. The method according to claim 5, wherein the detection of a protein level is by Western blot analysis.

7. The method according to claim 1, wherein the comparison of protein levels between the protein of Step (a) and the same protein in a control tumor cell sample is performed by measuring and comparing the intensity of bands according to Western blot analysis with naked eyes or image scanning analysis.

8. The method according to claim 1, wherein the premature senescence is one or more selected from irradiation-induced senescence and anticancer drug-induced senescence.

9. The method according to claim 8, wherein the anticancer drug is selected from camptothecin, etoposide, doxorubicin, and any combination thereof

10. The method according to claim 1, wherein the control tumor cell sample is tumor cells prior to application of irradiation or an anticancer drug, and the tumor sample is tumor cells after application of irradiation or an anticancer drug.

11. The method according to claim 10, wherein the control tumor cell sample is tumor cells prior to application of irradiation, and the tumor sample is tumor cells 3 to 5 days after application of irradiation.

12. The method according to claim 10, wherein the control tumor cell sample is tumor cells prior to application of an anticancer drug, and the tumor sample is tumor cells 3 to 5 days after application of an anticancer drug.

13. The method according to claim 1, wherein the tumor cells are selected from breast cancer cells, lung cancer cells, colon cancer cells, and any combination thereof

14. A method for detecting a premature senescence state of tumor cells in a human subject, comprising:
(a) detecting a level of a target protein in a tumor sample obtained from the human subject, wherein the protein is one or more selected from the group consisting of eukaryotic elongation factor 1 alpha 1 (eEF1A1) and cathepsin D (CD); and
(b) comparing a level of the target protein of Step (a) and a level of the same protein in a control tumor cell sample to determine one or more of decrease of eEF1A1 level and increase of CD level in the tumor sample.

15. The method according to claim 14, wherein the detection of a protein level is by antibodies that specifically bind to the protein.

16. The method according to claim 14, wherein the premature senescence is one or more selected from irradiation-induced senescence and anticancer drug-induced senescence.

17. A method for the diagnosis or prognosis of a premalignant or malignant state of tumor cells in a human subject, comprising:
(a) detecting a level of a target protein in a tumor sample obtained from the human subject, wherein the protein is one or more selected from the group consisting of eukaryotic elongation factor 1 alpha 1 (eEF1A1) and cathepsin D (CD); and
(b) comparing a level of the target protein of Step (a) and a level of the same protein in a control tumor cell sample to determine one or more of decrease of eEF1A1 level and increase of CD level in the tumor sample.

18. The method according to claim 17, wherein the detection of a protein level is by antibodies that specifically bind to the protein.

19. The method according to claim 17, wherein the detection of a protein level is by Western blot analysis.

20. A method of screening for a premature senescence-inducing agent in a tumor cell, comprising:
(a) collecting a first tumor cell sample before exposing to a candidate premature senescence-inducing agent;
(b) collecting a second tumor cell sample after exposing to a candidate premature senescence-inducing agent;
(c) detecting levels of one or more of CD and eEF1A1 in the first and second samples;
(d) determining whether a level of CD increases and/or whether a level of eEF1A1 decreases; and
(e) identifying a candidate agent exhibiting one or more of an increased CD level and a decreased eEF 1A1 level, as a premature senescence-inducing agent.

21. The method according to claim 20, wherein the detection of a protein level is by antibodies that specifically bind to the protein.

22. A method of screening a potential candidate of cancer therapy in a human subject, the method comprising:
(a) collecting a first tumor cell sample before exposing to a candidate premature senescence-inducing agent;
(b) collecting a second tumor cell sample after exposing to a candidate premature senescence-inducing agent;
(c) detecting levels of one or more of CD and eEF1A1 in the first and second samples;
(d) determining whether a level of CD increases and/or whether a level of eEF1A1 decreases; and
(e) identifying a potential cancer therapy agent exhibiting one or more of an increased CD level and a decreased eEF1A1 level, as a premature senescence-inducing agent.

23. The method according to claim 22, wherein the detection of a protein level is by antibodies that specifically bind to the protein.

24. A kit for detecting premature senescence in tumor cells, comprising antibodies directed against eEF1A1 and antibodies directed against CD.


SEQUENCE LISTING (2 sequences)

SEQ ID NO: 1 - DNA, 2117 bp, Homo sapiens
  mRNA (1)..(2117): Homo sapiens cathepsin D, mRNA(cDNA clone), complete cds, GenBank BC016320.
aagcgcacggcctcggcgaccctctccgacccggccgccgccgccatgcagccctccagccttctgccgctcgccctctgcctgctggctgcacccgcctccgcgctcgtcaggatcccgctgcacaagttcacgtccatccgccggaccatgtcggaggttgggggctctgtggaggacctgattgccaaaggccccgtctcaaagtactcccaggcggtgccagccgtgaccgaggggcccattcccgaggtgctcaagaactacatggacgcccagtactacggggagattggcatcgggacgcccccccagtgcttcacagtcgtcttcgacacgggctcctccaacctgtgggtcccctccatccactgcaaactgctggacatcgcttgctggatccaccacaagtacaacagcgacaagtccagcacctacgtgaagaatggtacctcgtttgacatccactatggctcgggcagcctctccgggtacctgagccaggacactgtgtcggtgccctgccagtcagcgtcgtcagcctctgccctgggcggtgtcaaagtggagaggcaggtctttggggaggccaccaagcagccaggcatcaccttcatcgcagccaagttcgatggcatcctgggcatggcctacccccgcatctccgtcaacaacgtgctgcccgtcttcgacaacctgatgcagcagaagctggtggaccagaacatcttctccttctacctgagcagggacccagatgcgcagcctgggggtgagctgatgctgggtggcacagactccaagtattacaagggttctctgtcctacctgaatgtcacccgcaaggcctactggcaggtccacctggaccaggtggaggtggccagcgggctgaccctgtgcaaggagggctgtgaggccattgtggacacaggcacttccctcatggtgggcccggtggatgaggtgcgcgagctgcagaaggccatcggggccgtgccgctgattcagggcgagtacatgatcccctgtgagaaggtgtccaccctgcccgcgatcacactgaagctgggaggcaaaggctacaagctgtccccagaggactacacgctcaaggtgtcgcaggccgggaagaccctctgcctgagcggcttcatgggcatggacatcccgccacccagcgggccactctggatcctgggcgacgtcttcatcggccgctactacactgtgtttgaccgtgacaacaacagggtgggcttcgccgaggctgcccgcctctagttcccaaggcgtccgcgcgccagcacagaaacagaggagagtcccagagcaggaggcccctggcccagcggcccctcccacacacacccacacactcgcccgcccactgtcctgggcgccctggaagccggcggcccaagcccgacttgctgttttgttctgtggttttcccctccctgggttcagaaatgctgcctgcctgtctgtctctccatctgtttggtgggggtagagctgatccagagcacagatctgtttcgtgcattggaagaccccacccaagcttggcagccgagctcgtgtatcctggggctcccttcatctccagggagtcccctccccggccctaccagcgcccgctgggctgagcccctaccccacaccaggccgtcctcccgggccctcccttggaaacctgccctgcctgagggcccctctgcccagcttgggcccagctgggctctgccaccctacctgttcagtgtcccgggcccgttgaggatgaggccgctagaggcctgaggatgagctggaaggagtgagaggggacaaaacccaccttgttggagcctgcagggtggtgctgggactgagccagtcccaggggcatgtattggcctggaggtggggttgggattgggggctggtgccagccttcctctgcagctgacctctgttgtcctccccttgggcggctgagagccccagctgacatggaaatacagttgttggcctccggcctcccctcaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 1668 bp, Homo sapiens
  mRNA (1)..(1668): Homo sapiens eukaryotic translation elongation factor 1 alpha 1, mRNA (cDNA clone), complete cds, GenBank BC071619
gccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaaaaaaaaaaaaaa